(19) United States
(12) Patent Application Publication
(10) Pub. No.: US 2011/0015523 A1
(43) Pub. Date: Jan. 20, 2011

(54) ULTRASOUND OBSERVATION APPARATUS

(75) Inventor: Tomohiro SABATA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/825,970

(22) Filed: Jun. 29, 2010

Related U.S. Application Data
(63) Continuation of application No. PCT/JP2009/068592, filed on Oct. 29, 2009.

(30) Foreign Application Priority Data
Oct. 31, 2008 (JP) 2008-282035

(51) Int. Cl. A61B 8/14

(52) U.S. Cl. 600/443

(57) ABSTRACT

An ultrasound observation apparatus has an ultrasound probe or an ultrasound endoscope manually moved relative to a subject, and displays a plurality of ultrasound tomographic images in time sequence with the movement. The ultrasound observation apparatus has a control section which, when a first display range is selected, performs control so that images are displayed in a first number of displayed frames per stroke time, which, when a second display range is selected, performs control so that the number of displayed frames per stroke time is smaller than the first number of displayed frames, and which, when a manual scanning mode is selected, performs control so that a predetermined number of frames per stroke time are displayed regardless of whether the display range is the first display range or the second display range.

### CROSS REFERENCE TO RELATED APPLICATION

This application is a continuation application of PCT/JP2009/068592 filed on Oct. 29, 2009 and claims benefit of Japanese Application No. 2008-282035 filed in Japan on Oct. 31, 2008, the entire contents of which are incorporated herein by this reference.

### BACKGROUND OF THE INVENTION

1. Field of the Invention

The present invention relates to an ultrasound observation apparatus and, more particularly, to an ultrasound observation apparatus capable of manually obtaining a plurality of ultrasound tomographic images.

2. Description of the Related Art

Ultrasound observation apparatuses have been widely used as an apparatus which repeatedly sends ultrasound pulses to a living tissue from an ultrasound transducer, receives waves of an echo signal formed by ultrasound pulses reflected from the living tissue and displays an ultrasound tomographic image of a subject.

In recent years, ultrasound observation apparatuses which produce a three-dimensional image from ultrasound tomographic image data have also been proposed. In particular, an apparatus having means for detecting the position and the orientation of a distal end portion of an electronic-scanning-type ultrasound probe for the purpose of producing a three-dimensional image, as disclosed in Japanese Patent Application Laid-Open Publication No. 2003-180697, has also been proposed.

In the apparatus according to the proposition, a magnetic field generation element is provided in the distal end portion of the probe, while a detection element for detecting a magnetic field generated from the magnetic field generation element is provided outside a subject. The position and the orientation of an electronic radial scanning plane perpendicular to the probe axis are detected on the basis of the magnetic field obtained by the detection element. Voxel data is generated on the basis of information on the detected position and orientation, thus enabling display of a distortion-free three dimensional image.

Ultrasound observation apparatuses include mechanical scanning type of apparatuses which perform scanning in a body cavity by mechanically rotating a distal end portion having an ultrasound vibration element, as well as electronic scanning types of apparatuses.

An endoscopic ultrasound observation apparatus EU-M2000 manufactured and sold by the applicant of the present application is of a mechanical scanning type and capable of producing a three-dimensional image by so-called manual-draw scanning. In this apparatus, no element for detecting the position and orientation is provided in a distal end portion of a probe.

Manual-draw scanning is performed, for example, by a method shown in FIG. 16. FIG. 16 is a diagram for explaining a case where an operator obtains image data by performing manual-draw scanning with a probe. The operator inserts a distal end portion of a probe to a desired position and performs manual scanning by drawing the probe so that the probe is returned toward the operator. Data on a plurality of tomographic images is thereby obtained. In the case shown in FIG. 16, the distal end portion is drawn from a position A to a position B via a position C.

For example, the operator sets the range of display of an ultrasound tomographic image to 12 cm, cancels a freeze and performs manual-draw scanning with the probe from the position A to the position B. When the probe reaches the position B, the image is frozen.

In a case where the operator seeing the tomographic image temporarily obtained wants to see a particular portion, e.g., a tumor portion by enlarging the portion, he or she changes the display range, for example, to 3 cm and again performs manual-draw scanning with the probe from the position A to the position B by the same procedure as that described above. As a result, the particular portion is displayed by being enlarged and the operator can make a detailed observation.

An application of the functions of the mechanical-scanning-type apparatus capable of producing a three-dimensional image as described above to an ultrasound observation apparatus to which an electronic-scanning-type probe is connected is also conceivable. An apparatus of an electronic scanning type is also capable of producing a three-dimensional image if manual-draw scanning is performed, as is the above-described mechanical-scanning-type apparatus.

Ordinarily, in a mechanical-scanning-type ultrasound observation apparatus, the distal end portion of the probe is mechanically rotated and the frame rate is fixed because of a structural problem such as a mechanical accuracy problem due to the mechanical rotation. FIG. 17 is a diagram showing an example of a 3D display of tomographic images obtained by a mechanical-scanning-type apparatus when the display range is 12 cm. FIG. 18 is a diagram showing an example of a 3D display of tomographic images obtained by the mechanical-scanning-type apparatus when the display range is 3 cm. FIGS. 17 and 18 show examples of displays of ultrasound tomographic images produced on a monitor screen. A tomographic image along a scanning plane perpendicular to the probe axis is shown on the left-hand side, while a tomographic image along the probe axis direction is shown on the right-hand side.

For example, even in a case where a tomographic image (FIG. 18) of a subject is obtained by changing the display range to 3 cm after seeing a tomographic image of the subject (FIG. 17) by setting the display range to 12 cm, the stroke time when manual-draw scanning is performed from a position A to a position B in the tomographic image along the probe axis direction on the right-hand side is constant because the number of frames displayed on the screen and the frame rate are constant on the monitor screen (each of the stroke time in the case shown in FIG. 17 and the stroke time in the case shown in FIG. 18 is 12 seconds).

That is, since the stroke time=(the number of frames/the frame rate), the stroke time for a section along the manual-draw direction in FIG. 17 and the stroke time of a section along the manual-draw direction in FIG. 18 are equal to each other.

Therefore, the operator may perform manual drawing at a fixed speed (the speed at which the probe is drawn from the position A to the position B) even when changing the display range (for example, from 12 cm to 3 cm).

On the other hand, in the case of an electronic-scanning-type ultrasound observation apparatus, the corresponding scanning is electronically performed. Therefore, the frame rate is changed according to the display range. FIG. 19 shows an example of a 3D display of tomographic images obtained by an electronic-scanning-type apparatus when the display range is 12 cm. FIG. 20 shows an example of a 3D display of tomographic images obtained by the electronic-scanning-type apparatus when the display range is 3 cm.

### SUMMARY OF THE INVENTION

According to the present invention, there is provided an ultrasound observation apparatus which has an ultrasound probe or an ultrasound endoscope manually moved relative to a subject, and which displays a plurality of ultrasound tomographic images in time sequence with the movement, the apparatus including a control section which, when a first display range is selected, performs control so that images are displayed in a first number of displayed frames per the stroke time, which, when a second display range is selected, performs control so that the number of displayed frames per the stroke time is smaller than the first number of displayed frames, and which, when a manual scanning mode is selected, performs control so that a predetermined number of frames per the stroke time are displayed regardless of whether the display range is the first display range or the second display range.

### BRIEF DESCRIPTION OF THE DRAWINGS

FIG. 1 is a block diagram showing the entire configuration of an ultrasound diagnostic apparatus in a first embodiment of the present invention;

FIG. 2 is a block diagram of a portion of the ultrasound diagnostic apparatus in FIG. 1 relating to the operation of the first embodiment;

FIG. 3 is a flowchart showing an example of the flow of the entire processing in the ultrasound diagnostic apparatus in the first embodiment of the present invention;

FIG. 4 is a flowchart showing an example of the flow of part of frame rate fixing processing in step S2 in FIG. 3;

FIG. 5 is a timing chart of a freeze control signal, a frame sync signal F_sync, a TX trigger and a frame rate control signal FRM_CNT in the related art;

FIG. 6 is a flowchart showing an example of the flow of processing for frame rate fixing control in a signal processing section in the first embodiment of the present invention;

FIG. 7 is a flowchart showing an example of the flow of frame rate fixing control in an electronic-side timing controller in the first embodiment of the present invention;

FIG. 8 is a flowchart showing an example of the flow of processing for frame rate fixing control in a beam former section in the first embodiment of the present invention;

FIG. 9 is a timing chart of a freeze control signal, a frame sync signal F_sync, a TX trigger and a frame rate control signal FRM_CNT in the first embodiment of the present invention;

FIG. 10 is a flowchart showing an example of the flow of processing for frame rate fixing control in a video processing section according to a second embodiment of the present invention;

FIG. 11 is a flowchart showing details of frame data output processing in step S52 in FIG. 10;

FIG. 12 is a diagram for explaining output and discarding of frame data by processing shown in FIGS. 10 and 11;

FIG. 13 is a flowchart showing an example of the flow of the entire processing in an ultrasound diagnostic apparatus in a third embodiment of the present invention;

FIG. 14 is a diagram showing an example of an input dialog for input of a stroke time according to the third embodiment of the present invention;

FIG. 15 is a flowchart showing an example of the flow of processing for frame rate fixing control according to a fourth embodiment of the present invention;

FIG. 16 is a diagram for explaining a case where an operator obtains image data by performing manual-draw scanning with a probe;

FIG. 17 is a diagram showing an example of a 3D display of tomographic images obtained by a mechanical-scanning-type apparatus when the display range is 12 cm;

FIG. 18 is a diagram showing an example of a 3D display of tomographic images obtained by the mechanical-scanning-type apparatus when the display range is 3 cm;

FIG. 19 is a diagram showing an example of a 3D display of tomographic images obtained by an electronic-scanning-type apparatus when the display range is 12 cm; and

FIG. 20 is a diagram showing an example of a 3D display of tomographic images obtained by the electronic-scanning-type apparatus when the display range is 3 cm.

### DETAILED DESCRIPTION OF THE PREFERRED EMBODIMENT(S)

Embodiments of the present invention will be described with reference to the drawings.

### First Embodiment

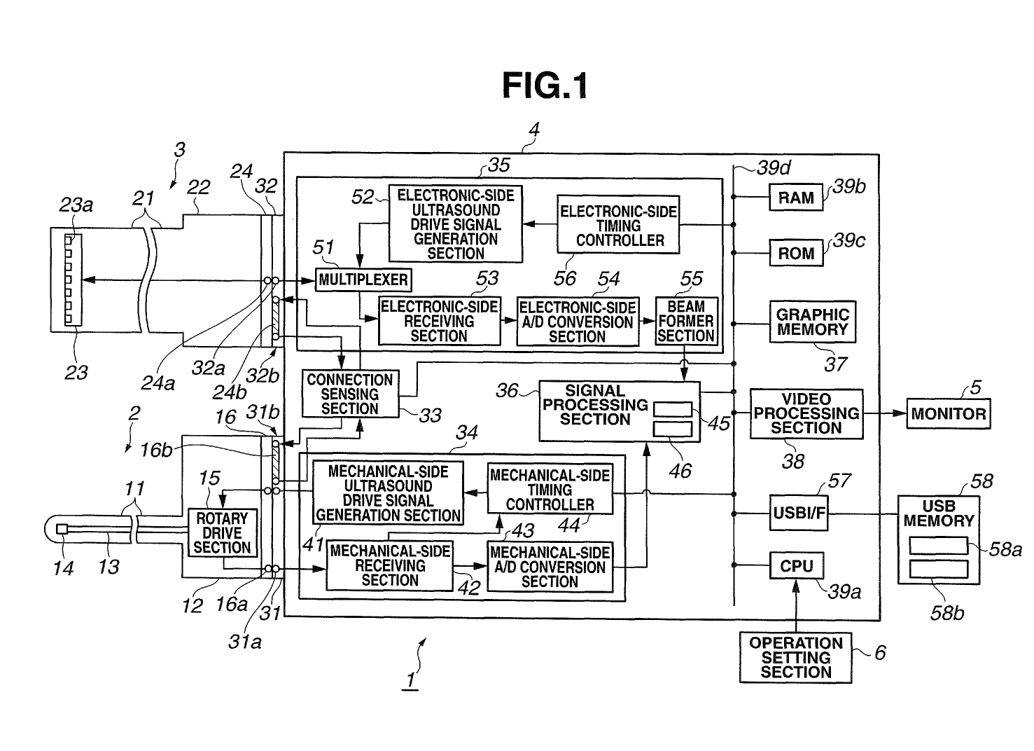

FIG. 1 is a block diagram showing the entire configuration of an ultrasound diagnostic apparatus in a first embodiment of the present invention. As shown in FIG. 1, an ultrasound diagnostic apparatus 1 in the first embodiment is configured to have a mechanical-scanning-type ultrasound probe 2, an electronic-scanning-type ultrasound endoscope 3 and an ultrasound observation apparatus 4. A monitor 5 and an operation setting section 6 are connected to the ultrasound observation apparatus 4.

The ultrasound observation apparatus 4 is constructed to have each of the mechanical-scanning-type ultrasound endoscope or ultrasound probe (ultrasound probe in this specification) 2 and the electronic-scanning-type ultrasound endoscope 3 detachably attached thereto. The ultrasound observation apparatus 4 obtains echo signals from the ultrasound probe 2 and the ultrasound endoscope 3, thereby forms an ultrasound tomographic image and displays the ultrasound tomographic image on the monitor 5.

The following description of each of the embodiments is made by illustrating an ultrasound endoscope as an electronic-scanning-type apparatus by way of example. However, the manually operated electronic-scanning-type apparatus described below may not be an endoscope but an ordinary electronic-scanning-type ultrasound probe.

The mechanical-scanning-type ultrasound probe 2 has an insertion portion 11 formed in an elongated shape such that the insertion portion 11 can be easily inserted into an internal portion of a subject or the like, and an operation portion 12 provided at a rear end of the insertion portion 11. The mechanical-scanning-type ultrasound probe 2 has an ultrasound transducer 14 fixed at a distal end side in a flexible shaft 13 inserted in the insertion portion 11.

A rear end of the flexible shaft 13 is connected to a rotary drive section 15 provided in the operation portion 12. The rotary drive section 15 rotates the flexible shaft 13 by a motor not shown in the figure, thereby mechanically rotating and driving the ultrasound transducer 14. In the rotary drive section 15, a rotational position detection section such as an encoder not shown in the figure is provided. A space surrounding the ultrasound transducer 14 is filled with an ultrasound propagation medium not shown in the figure for transmitting (propagating) ultrasound.

In the operation portion 12, a mechanical-side connector 16 detachably connected to the ultrasound observation apparatus 4 is provided. The mechanical-side connector 16 has a mechanical-side electrical contact portion 16a to which a signal line from the rotary drive section 15 is connected. In the mechanical-side connector 16, a mechanical-side connection sensing projection portion 16b for sensing through a connection sensing section 33 described below the connection of the mechanical-scanning-type ultrasound probe 2 to the ultrasound observation apparatus 4 is also provided.

The ultrasound transducer 14 of the mechanical-scanning-type ultrasound probe 2 is electrically connected to the ultrasound observation apparatus 4 through a signal line passed through the interior of the flexible shaft 13 when the mechanical-side connector 16 is connected to the ultrasound observation apparatus 4.

On the other hand, the electronic-scanning-type ultrasound endoscope 3 has an insertion portion 21 formed in an elongated shape such that the insertion portion 21 can be easily inserted into an internal portion of a subject or the like, and an operation portion 22 provided at a rear end of the insertion portion 21. An ultrasound transducer 23 is disposed in a distal end portion in the insertion portion 21. The ultrasound transducer 23 is formed by arranging a plurality of transducer elements 23a.

In the operation portion 22, an electronic-side connector 24 detachably connected to the ultrasound observation apparatus 4 is provided. The electronic-side connector 24 has an electrical contact portion 24a to which a signal line from the ultrasound transducer 23 is connected. In the electronic-side connector 24, an electronic-side connection sensing projection portion 24b for sensing through the connection sensing section 33 described below the connection of the electronic-scanning-type ultrasound endoscope 3 to the ultrasound observation apparatus 4 is also provided. The ultrasound transducer 23 of the electronic-scanning-type ultrasound endoscope 3 is electrically connected to the ultrasound observation apparatus 4 through the signal line when the electronic-side connector 24 is connected to the ultrasound observation apparatus 4.

The electronic-scanning-type ultrasound endoscope 3 is also connected to a light source unit and a video processor not shown in the figure. The ultrasound endoscope 3 has in a distal end portion in the insertion portion 21 an illumination optical system, an objective optical system and an image pickup section not shown in the figure. The ultrasound endoscope 3 illuminates through the illumination optical system the interior of a body cavity with illumination light supplied from the light source unit, takes in light reflected from the illuminated interior body cavity as a subject image through the objective optical system, and picks up an image through the image pickup section. An image pickup signal from the image pickup section is supplied to the video processing section 38 to undergo signal processing. A standard video signal is thereby produced and is outputted to an optical image monitor (not shown in the figure).

Further, the ultrasound endoscope 3 has a treatment instrument insertion channel not shown in the figure. The mechanical-scanning-type ultrasound probe 2 can be inserted into a body cavity by being inserted in the treatment instrument insertion channel in the ultrasound endoscope 3 and caused to project from an opening of this channel.

The ultrasound observation apparatus 4 has a mechanical-side connector receiving portion 31 as a first connection portion to which the mechanical-side connector 16 of the mechanical-scanning-type ultrasound probe 2 is detachably connected, and an electronic-side connector receiving portion 32 as a second connection portion to which the electronic-side connector 24 of the electronic-scanning-type ultrasound endoscope 3 is detachably connected.

In the mechanical-side connector receiving portion 31, a receiving-side electrical contact portion 31a to be brought into conductive contact with the mechanical-side electrical contact portion 16a of the mechanical-side connector 16, and a mechanical-side fitting recess 31b in which the mechanical-side connection sensing projection portion 16b of the mechanical-side connector 16 is fitted, are provided.

On the other hand, in the electronic-side connector receiving portion 32, a receiving-side electrical contact portion 32a to be brought into conductive contact with the electrical contact portion 24a of the electronic-side connector 24, and an electronic-side fitting recess 32b in which the electronic-side connection sensing projection portion 24b of the electronic-side connector 24 is fitted, are provided.

The ultrasound observation apparatus 4 also has, as a plurality of circuit sections, the connection sensing section 33, a mechanical-system transducer echo signal detection section (hereinafter referred to as “mechanical-system echo signal detection section”) 34, an electronic-system transducer echo signal detection section (hereinafter referred to as “electronic-system echo signal detection section”) 35, a signal processing section 36, a graphic memory 37, the video processing section 38, a CPU 39a, which is a central processing unit, a RAM 39b, a ROM 39c and a USB (universal serial bus) interface (I/F) 57. These circuit sections are electrically connected to each other through a bus 39d such as a PCI bus.

The connection sensing section 33 is electrically connected to the mechanical-side and electronic-side fitting portions 31b and 32b. When the mechanical-side and electronic-side connection sensing projection portions 16b and 24b are respectively fitted in these mechanical-side and electronic-side fitting recesses 31b and 32b, conduction is caused between each of the pairs of contacts of the mechanical-side and electronic-side fitting recesses 31b and 32b. The connections of the mechanical-side connector 16 and the electronic-side connector 24 are then sensed. The connection sensing section 33 outputs a connection sensing signal to the CPU 39a through the bus 39d.

The mechanical-system echo signal detection section 34 sends ultrasound pulses from the ultrasound transducer 14 incorporated in the ultrasound probe 2 to a living tissue and detects an echo signal obtained by receiving ultrasound pulses reflected from the living tissue.

The electronic-system echo signal detection section 35 sends ultrasound pulses from the ultrasound transducer 23 incorporated in the electronic-scanning-type ultrasound endoscope 3 to a living tissue and detects an echo signal obtained by receiving ultrasound pulses reflected from the living tissue.

The signal processing section 36 performs signal processing on the echo signals from the mechanical-system echo signal detection section 34 and the electronic-system echo signal detection section 35. The signal processing section 36 is a circuit including an FPGA (field programmable gate array) and a DSP (digital signal processor) and capable of executing a piece of software. The CPU 39a performs polar coordinate conversion of the echo signal on which signal processing has been performed by the signal processing section 36, thereafter performs image processing on the signal to obtain a display signal, and outputs the display signal to the video processing section 38.

The signal processing section 36 includes a flash ROM 45 for FPGA configuration and a flash ROM 46 for DSP configuration. More specifically, these flash ROMS 45 and 46 are mounted on a circuit board for the signal processing section 36 together with the FPGA and the DSP. In the flash ROMs 45 and 46, groups of configuration data for the FPGA and the DSP are respectively stored. Data for Log compression processing or the like is also stored in the flash ROMs.

The video processing section 38 performs signal processing on a display signal processed by the CPU 39a, performs scan conversion of the signal and outputs the signal to the monitor 5 to display an ultrasound tomographic image on the display screen of the monitor 5.

The graphic memory 37 receives and stores image data in the echo signal from the signal processing section 36 and temporarily stores the echo signal on a frame-by-frame basis at the time of signal processing by the video processing section 38. In the ROM 39c, programs for controlling various operations in the ultrasound observation apparatus 4 are stored.

The CPU 39a controls the entire ultrasound observation apparatus 4 on the basis of the programs stored in the ROM 39c. The CPU 39a controls the mechanical-system echo signal detection section 34 and the electronic-system echo signal detection section 35 on the basis of a setting command inputted from a setting button or the like in the operation setting section 6 so as to obtain an ultrasound tomographic image by controlling one of the mechanical-scanning-type ultrasound probe 2 and the electronic-scanning-type ultrasound endoscope 3.

The CPU 39a controls a mechanical-side timing controller 44 or an electronic-side timing controller 56 described below according to whether the present mode is a mechanical mode with the ultrasound probe 2 or an electronic mode with the ultrasound endoscope 3, and outputs scanning discrimination information to the signal processing section 36 according to whether the present mode is a mechanical mode with the ultrasound probe 2 or an electronic mode with the ultrasound endoscope 3.

To the USB I/F 57, a USB memory 58 can be connected. In the USB memory 58, configuration data 58a for the signal processing section 36 and an application program 58b for writing the configuration data 58a to the flash ROMs 45 and 46 in the signal processing section 36 are stored.

When the configuration data for the FPGA or the DSP in the signal processing section 36 are to be rewritten, that is, the details of processing in the signal processing section 36 are to be changed, the USB memory 58 is inserted in the USB I/F 57 to execute the application program 58b by the CPU 39a. The application program 58b rewrites the contents of the flash ROMs 45 and 46 by using the configuration data 58a written in the USB memory 58a. This rewriting is performed by transferring data through the bus 39d, which is a common bus such as a PCI bus in the ultrasound observation apparatus 4.

Thus, the configuration data for the FPGA and the DSP in the signal processing section 36 can be rewritten through the USB I/F 57 and the bus 39d by using the application program 59b and the configuration data 58a stored in the external USB memory 58 separate from the ultrasound observation apparatus 4. Therefore, when the ultrasound observation apparatus 4 is started up, the FPGA and the DSP in the signal processing section 36 are configured on the basis of the rewritten configuration data in the flash ROMs 45 and 46, thereby determining details of processing in the signal processing section 36.

Further, rewriting of various sorts of filter information for image processing other than the configuration data can also be performed by using the USB memory 58a.

Also, the ultrasound observation apparatus 4 is configured so that after the completion of configuration of each of the FPGA and the DSP at the time of powering on, status information is written to a predetermined register to enable confirmation of the completion of the configuration.

More specifically, each of the programmable devices including the FPGA and the like transmits to a status sensing section (not shown in the figure) predetermined statue information, e.g., bits to a predetermined register after the completion of configuration. The status sensing section itself may be a programmable device. In the status sensing section, each group of predetermined status information is written to the predetermined register.

When the application program in the ultrasound observation apparatus 4 is executed, the application program checks the content of each register in the status sensing section to determine whether or not each of the FPGA and so on has been correctly configured. If the predetermined status information is not written in the predetermined register, it is determined that the corresponding one of the devices including the FPGA has not been correctly configured. Predetermined error notification or display processing is then performed. If an error indication is provided on the monitor 5, a user can easily know in which device failure to correctly perform configuration has occurred.

The configuration data for each programmable device further includes version information. Further, the above-mentioned filter information for image processing also includes version information. These groups of version information are written to the flash ROMs 45 and 46 and can be checked by being displayed on the screen of the monitor 5 by a predetermined operation performed by a user.

There is also version information about processing in a beam former section 55. This version information is embedded on a circuit board for the beam former section 55 and can also be displayed on the monitor 5.

Also, in STC processing, the gain of an amplifier with respect to the echo signal is changed according to the depth. The ultrasound observation apparatus 4 is configured so that for the values of corrections to the gain, with respect to several points on an STC curve, depth data, amplifier gain values and gradient values between the points on the STC curve are set in registers in the signal processing section 36 by a piece of application software executed by the CPU 39a. The signal processing section 36 computes (interpolates) STC values between the set points from the values of the set points and performs STC processing on the echo signal by using the computed STC values.

That is, on the basis of data on correction values at several points given from the application software, the signal processing section 36 performs STC processing on the original echo signal. Accordingly, when the display range is changed, for example, from 12 cm to 2 cm, the signal processing section 36 generates 2 cm data not by thinning out 12 cm data but by performing STC processing on the echo signal (the original data before thinning out). As a result, the gradation of the displayed image data is made smooth.

Further, if an operation using the Doppler effect for blood flow display in the electronic scanning system can be performed, it is desirable to set lower the gain of an extremely shallow portion, i.e., a near-point portion, in the STC curve. More specifically, in the vicinity of the transducer, e.g., within a range of 2 mm, the intensity of the echo signal is so high that Doppler data cannot be correctly sensed and, therefore, it is preferable to set the value of the STC curve so as to limit the gain of the amplifier with respect to the echo signal for the near-point portion. Removal of noise components can be performed in this way.

Details of the internal configuration of the mechanical-system echo signal detection section 34 will next be described.

The mechanical-system echo signal detection section 34 has a mechanical-side ultrasound drive signal generation section 41, a mechanical-side receiving section 42, a mechanical-side A/D conversion section 43 and the mechanical-side timing controller 44.

The mechanical-side ultrasound drive signal generation section 41 generates and outputs, on the basis of a timing signal from the mechanical-side timing controller 44, ultrasound drive pulses for driving the ultrasound transducer 14 and a drive signal for driving the rotary drive section 15.

The mechanical-side receiving section 42 receives the echo signal from the ultrasound transducer 14 and performs analog signal processing.

More specifically, the mechanical-side receiving section 42 is configured of an amplifier which amplifies the echo signal and filters for preventing aliasing in the mechanical-side A/D conversion section 43: a LPF (low-pass filter) and a BPF (band-pass filter).

The mechanical-side A/D conversion section 43 performs processing for converting an analog signal obtained by analog signal processing performed by the mechanical-side receiving section 42 into a digital signal, and outputs the digital signal to the signal processing section 36. The mechanical-side timing controller 44 generates and outputs the timing signal to the mechanical-side ultrasound drive signal generation section 41 on the basis of control signals from the CPU 39a and the rotational position detection circuit (encoder or the like) provided in the rotary drive section 15 but not shown in the figure.

The mechanical-side timing controller 44 receives a rotational position detection signal from the rotational position detection section in the rotary drive section 15 through the mechanical-side receiving section 42, generates a sync signal in synchronization with the rotation of the ultrasound transducer 14 and outputs the sync signal to the signal processing section 36.

Details of the internal configuration of the electronic-system echo signal detection section 35 will next be described.

The electronic-system echo signal detection section 35 has a multiplexer 51, an electronic-side ultrasound drive signal generation section 52, an electronic-side receiving section 53, an electronic-side A/D conversion section 54, the beam former section 55 and the electronic-side timing controller 56.

The multiplexer 51 selects any ones of the plurality of transducer elements 23a of the ultrasound transducer 23, outputs ultrasound pulses from the electronic-side ultrasound drive signal generation section 52 to the corresponding transducer elements 23a, and outputs echo signals from the corresponding transducer elements 23a to the electronic-side receiving section 53.

The electronic-side ultrasound drive signal generation section 52 generates a plurality of ultrasound drive pulses for respectively driving the plurality of transducer elements 23a of the ultrasound transducer 23 on the basis of a timing signal from the electronic-side timing controller 56 and outputs the drive pulses through the multiplexer 51.

The electronic-side receiving section 53 receives echo signals from the plurality of transducer elements 23a of the ultrasound transducer 23 through the multiplexer 51 and performs analog signal processing on the received echo signals. The electronic-side receiving section 53 is configured of components including an amplifier, a BPF and an LPF corresponding to those of the mechanical-side receiving section 42 in the mechanical-system echo signal detection section 34.

The electronic-side A/D conversion section 54 performs processing for converting analog signals obtained by analog signal processing performed by the electronic-side receiving section 53 into digital signals and sequentially outputs the digital signals.

The beam former section 55 combines the digitized echo signals by delaying the echo signals according to drive of the plurality of transducer elements 23a on the basis of the timing signal from the electronic-side timing controller 56, and outputs the combined signal to the signal processing section 36.

The electronic-side timing controller 56 generates the timing signal under the control of the CPU 39a and outputs the timing signal to the electronic-side ultrasound drive signal generation section 52. The electronic-side timing controller 56 also outputs the generated timing signal to the beam former section 55. The electronic-side timing controller 56 generates a sync signal with which the echo signals combined by the beam former section 55 are synchronized, and outputs the sync signal to the signal processing section 36.

As described above, the signal processing section 36 performs signal processing on the echo signals from the mechanical-scanning-type ultrasound probe 2 and the electronic-scanning-type ultrasound endoscope 3 respectively obtained by the mechanical-system echo signal detection section 34 and the electronic-system echo signal detection section 35.

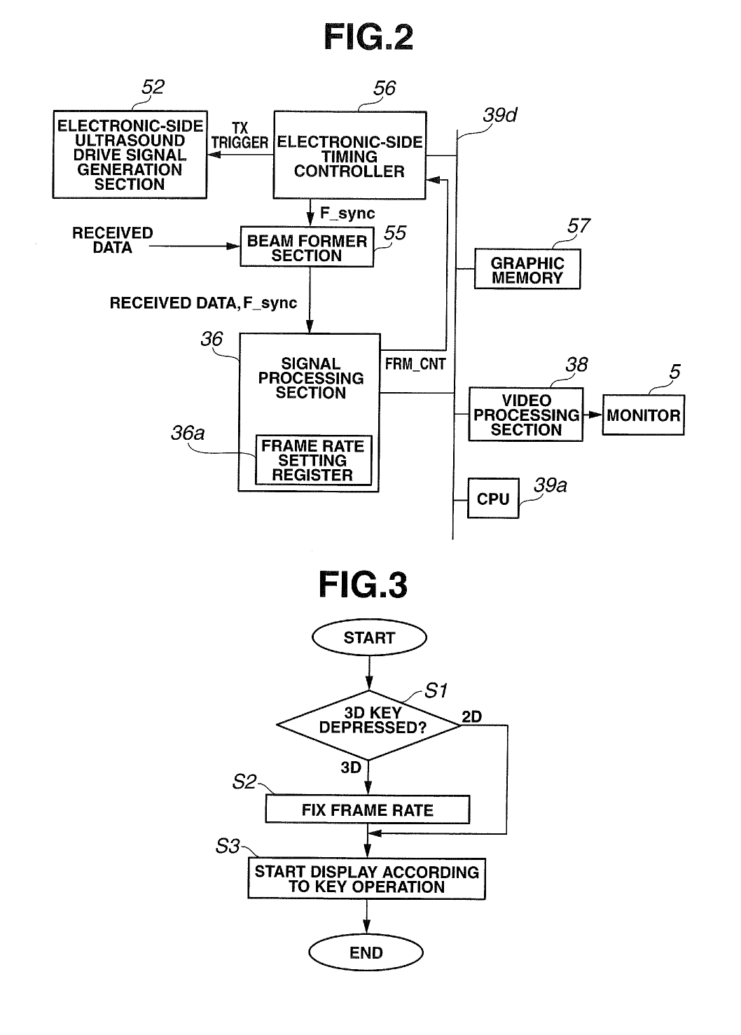

FIG. 2 is a block diagram of a portion of the ultrasound diagnostic apparatus 1 in FIG. 1 relating to the operation of the present embodiment. The signal processing section 36 has a frame rate setting register 36a. The operation of the circuit shown in FIG. 2 will be described together with the operation described below. Part of processings in the sections described below is realized by means of software.

While the CPU 39a is a processing section which executes processings for various functions by means of software, the electronic-side timing controller 56, the beam former section 55 and the signal processing section 36 are each a circuit including an FPGA and capable of executing a piece of software.

An operator who is a user using the ultrasound diagnostic apparatus 1 selects between use of the electronic-scanning-type ultrasound endoscope 3 and use of the mechanical-scanning-type ultrasound probe 2.

The mechanical scanning system is free from the above-described problem. Therefore, a case where the operator uses the electronic-scanning-type ultrasound endoscope 3 will be described below. When using the ultrasound endoscope 3, the operator presses a selecting switch not shown in the figure to select the electronic-scanning-type ultrasound endoscope 3. By this selection, the ultrasound diagnostic apparatus 1 enters the electronic mode in which processing in the case of the electronic scanning system is executed.

FIG. 3 is a flowchart showing an example of the flow of the entire processing in the ultrasound diagnostic apparatus 1 in the present embodiment.

The operator selects between producing a 2D display of an image to be displayed on the screen of the monitor 5 and producing a 3D display of the image by operating a predetermined button or the like on the operation setting section 6. A 2D display is produced in the mode in which an ordinary tomographic image is displayed. A 3D display is produced in the mode in which three-dimensional data, i.e., a plurality of ultrasound tomographic images, are obtained to display tomographic images such as shown in FIGS. 19 and 20. The operator selects 3D display and then manually moves the ultrasound endoscope 3 forward or rearward with respect to a subject. As described below, a plurality of ultrasound tomographic images are inputted in time sequence to the ultrasound observation apparatus 4 with the forward or rearward movement, and a display of images such as shown in FIGS. 19 and 20 is produced on the monitor 5, so that the operator can observe a target region of the subject.

Accordingly, the CPU 39a first determines which of a 2D key and a 3D key is depressed (step S1).

If the 3D key is depressed, the CPU 39a fixes the frame rate at a value set in advance (step S2). The process then advances to subsequent step S3. Thus, even in the electronic mode, the frame rate is fixed when scanning is manually performed.

If the 2D key is depressed, the process advances to subsequent step S3. In this case, because of the mode in which an ordinary ultrasound tomographic image is displayed, the frame rate is changed according to the display range or the like for example.

The CPU 39a then starts displaying according to a key operation (step S3).

In step S3, in the case of a 2D display, an ordinary tomographic image is displayed.

In step S3, in the case of a 3D display, the operator performs a predetermined key operation for obtaining a plurality of ultrasound tomographic images to cancel a freeze control signal (set the signal to LOW), and performs manual-draw scanning, which is manual scanning, to obtain a display such as shown in FIG. 19. More specifically, referring to FIG. 19, the operator starts moving the ultrasound endoscope 3 from the position A in such a manual manner that the ultrasound endoscope 3 is drawn toward the operator, and stops moving the ultrasound endoscope 3 at the position B, thus enabling a tomographic image (an image on the right-hand side of FIG. 19) according to the manual scan from the position A to the position B to be displayed on the screen of the monitor 5.

FIG. 19 shows an example of a display of two display views, i.e., a dual-plane view. The on-screen display shown in FIG. 19 is produced on the basis of three-dimensional image data obtained by manual-draw scanning, i.e., a plurality of ultrasound tomographic images. A view RD on the right-hand side is a section along the axial direction of the insertion portion 21 of the ultrasound endoscope 3. In the display shown in FIG. 19, when the operator designates a desired position P on the view RD, a tomographic image of a section perpendicular to the axial direction at the designated position P is displayed on a view LD on the left-hand side.

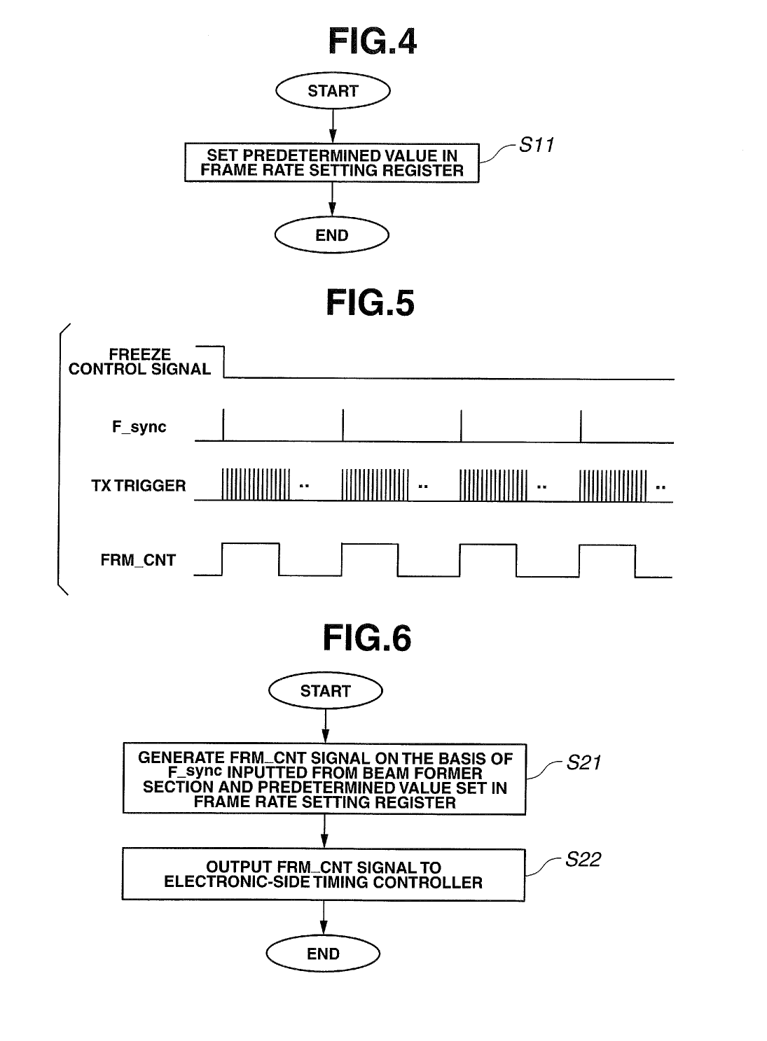

Processing in step S2 when the 3D key is depressed will be described. FIG. 4 is a flowchart showing the flow of part of frame rate fixing processing in step S2 in FIG. 3.

In step S2, more specifically, as shown in FIG. 4, the CPU 39a, which is a control section, sets a predetermined value in the frame rate setting register 36a in the signal processing section 36 (step S11).

For example, the CPU 39a as a control section sets a number of frames or a period corresponding to the number of frames as a predetermined value in the frame rate setting register 36a in the form of a piece of hardware. For example, a period of 143 milliseconds (ms) corresponding to 7 frames per second is set. This predetermined value may be a value set in advance or a set value changeable by a user.

The operations of a signal processing section, a beam former section and an electronic-side timing controller in an ultrasound diagnostic apparatus in the related art will be described.

FIG. 5 is a timing chart of a freeze control signal, a frame sync signal F_sync, a TX trigger and a frame rate control signal FRM_CNT in the related art. In the related art, the frame rate is changed according to the display range for example. Therefore, as shown in FIG. 5, when obtaining of three-dimensional data is started, the freeze control signal becomes LOW and the frame rate control signal FRM_CNT according to the frame rate is generated. The frame sync signal F_sync and the TX trigger are generated according to the frame rate control signal FRM_CNT. The TX trigger is a line sync signal.

In contrast, in the present embodiment, the ultrasound diagnostic apparatus 1 operates while the frame rate is fixed as described above. Processing in the signal processing section 36 in that case will be described below. FIG. 6 is a flowchart showing an example of the flow of processing for frame rate fixing control in the signal processing section 36 in the present embodiment.

First, the signal processing section 36, which is control means or a control section, generates a frame rate control signal FRM_CNT on the basis of a frame sync signal F_sync inputted from the beam former section 55 and a predetermined value set in the frame rate setting register 36a (step S21).

The signal processing section 36 then outputs the frame rate control signal FRM_CNT to the electronic-side timing controller 56 (step S22).

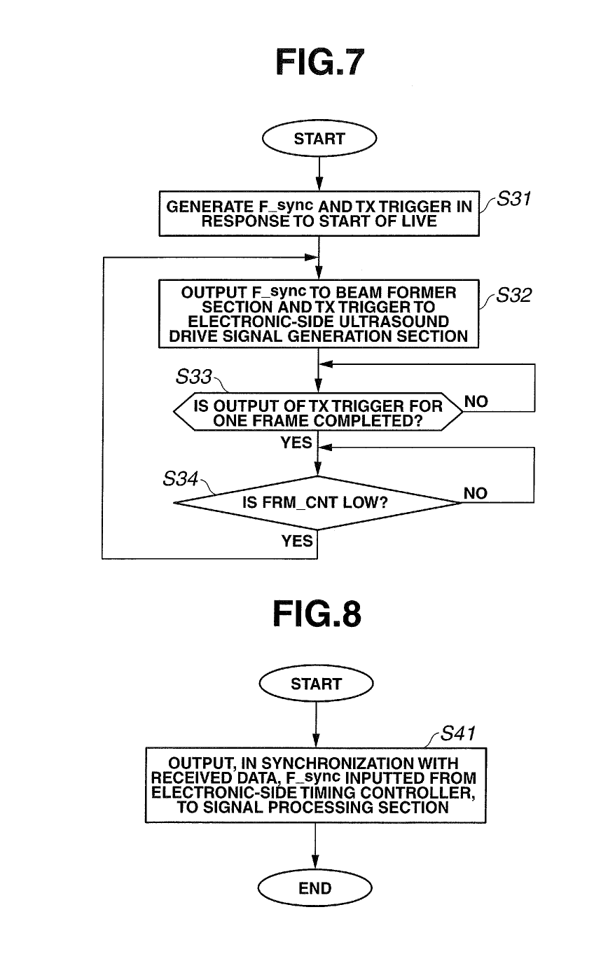

Processing in the electronic-side timing controller 56, which is control means or a control section, will next be described. FIG. 7 is a flowchart showing an example of the flow of frame rate fixing control in the electronic-side timing controller 56 in the present embodiment.

As shown in FIG. 7, the electronic-side timing controller 56 first generates the frame sync signal F_sync and a TX trigger in response to a start of display in step S3 (step S31).

The electronic-side timing controller 56 outputs the generated frame sync signal F_sync to the beam former section 55 and the generated TX trigger to the electronic-side ultrasound drive signal generation section 52 (step S32). The electronic-side ultrasound drive signal generation section 52 generates a transducer drive signal in synchronization with the inputted TX trigger and outputs the transducer drive signal to the multiplexer 51.

The electronic-side timing controller 56 determines whether or not output of the TX trigger corresponding to one frame has been completed (step S33). If output of the TX trigger has not been completed, that is, in the case of NO, the electronic-side timing controller 56 waits for the completion of output.

When output of the TX trigger corresponding to one frame is completed, the electronic-side timing controller 56 determines whether or not the frame rate control signal FRM_CNT has become LOW (step S34). If the frame rate control signal FRM_CNT is not LOW, that is, in the case of NO, the electronic-side timing controller 56 waits until the frame rate control signal FRM_CNT becomes LOW.

When the frame rate control signal FRM_CNT becomes LOW, that is, YES in step S34, the process returns to step S32.

FIG. 8 is a flowchart showing an example of the flow of processing for frame rate fixing control in the beam former section 55 in the present embodiment.

The beam former section 55, which is control means or a control section, synchronizes the frame sync signal F_sync inputted from the electronic-side timing controller 55 with received data and outputs the frame sync signal F_sync to the signal processing section 36 (step S41).

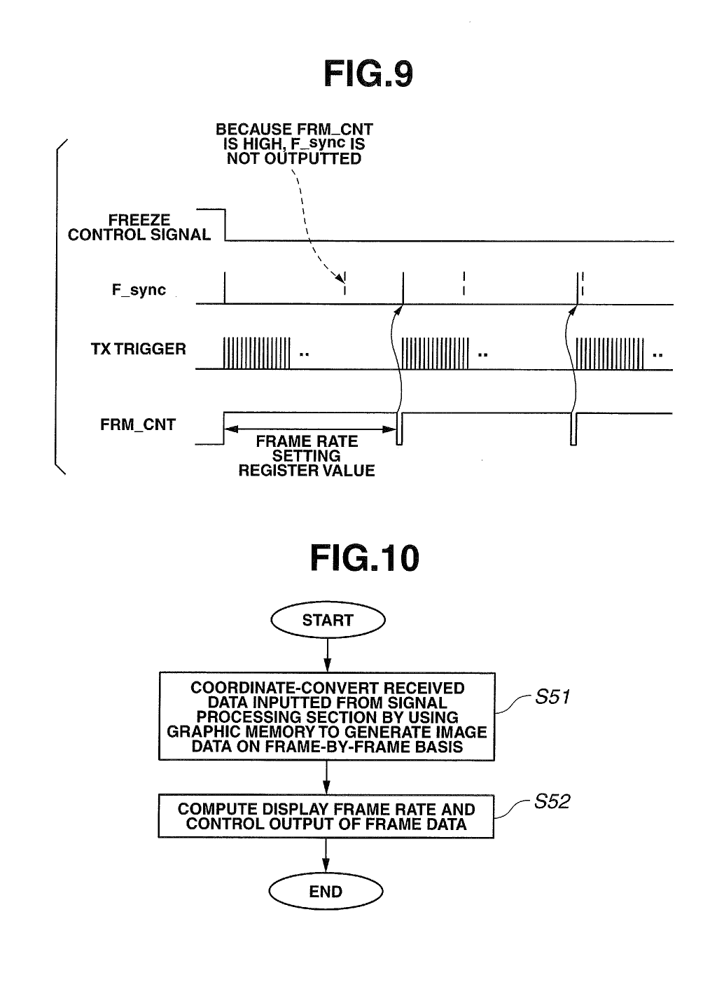

FIG. 9 is a timing chart of the freeze control signal, the frame sync signal F_sync, the TX trigger and the frame rate control signal FRM_CNT in the present embodiment. When obtaining of three-dimensional image data is started, the freeze control signal becomes LOW and the signal processing section 36 generates the frame rate control signal FRM_CNT according to the predetermined value set in the frame rate setting register 36a. The frame sync signal F_sync and the TX trigger are generated according to the frame rate control signal FRM_CNT.

As shown in FIG. 9, the frame sync signal F_sync and the TX trigger are generated when the frame rate control signal FRM_CNT becomes LOW. In other words, the electronic-side timing controller 56 is controlled so as not to output the frame sync signal F_sync and the TX trigger as long as FRM_CNT is HIGH.

Thus, in the manual scanning mode, control is performed by the control means so that the number of displayed frames of ultrasound tomographic images per stroke time is constant. Therefore, no complicated scanning is required in 3D displaying.

As described above, in the ultrasound observation apparatus in the present embodiment, the control means performs control by generating the frame sync signal on the basis of a set predetermined value so that the number of displayed frames of ultrasound tomographic images per stroke time is constant. Even with the electronic-type ultrasound endoscope, a user can obtain three-dimensional image data without performing any complicated operation such as changing the manual-draw speed according to the frame rate in the electronic system in the related art.

The stroke time is made constant at the time of producing a 3D display such as shown in FIG. 19. Therefore, the advantage of simplifying the configuration of the application program for display can also be obtained.

Even in a case where electronic-scanning-type ultrasound probes or ultrasound endoscopes have different frame rates, the above-described frame rate fixing control can be adapted for the ultrasound probes or the like having different frame rates. The above-described frame rate fixing control can therefore be applied to ultrasound probes or the like newly developed and having different frame rates.

### Second Embodiment

A second embodiment of the present invention will be described. An ultrasound diagnostic apparatus in the second embodiment has the same hardware configuration as that of the ultrasound diagnostic apparatus in the first embodiment. The same components as those of the ultrasound diagnostic apparatus in the first embodiment are indicated by the same reference characters, and the description for the same components will not be repeated. In the first embodiment, frame rate fixing control is realized by using the CPU 39a, the signal processing section 36 and the electronic-side timing controller 56. The second embodiment differs from the first embodiment in that frame rate fixing control is realized by means of a piece of software in the video processing section 38.

Also in the second embodiment, an operator can perform the above-described manual-draw scanning by depressing the 3D key to produce a display such as shown in FIGS. 19 and 20 on the monitor 5. As a result of manual-draw scanning by the operator, a plurality of tomographic images are obtained and accumulated in the graphic memory 37.

FIG. 10 is a flowchart showing an example of the flow of processing for frame rate fixing control in the video processing section according to the present embodiment.

Image data processed in the signal processing section 36 as a result of manual-draw scanning is transferred to and stored in the graphic memory 37.

The video processing section 38, which is control means or a control section, performs coordinate conversion of received data (i.e., image data) inputted from the signal processing section 36 by using the graphic memory 57 to generate image data on a frame-by-frame basis, i.e., frame data (step S51).

The video processing section 38 computes the display frame rate on the basis of a number of frames or a period set in advance by the operator and controls output of the frame data (step S52). The display frame rate may be set to a frame rate set in advance, for example, to a frame rate corresponding to the maximum display range.

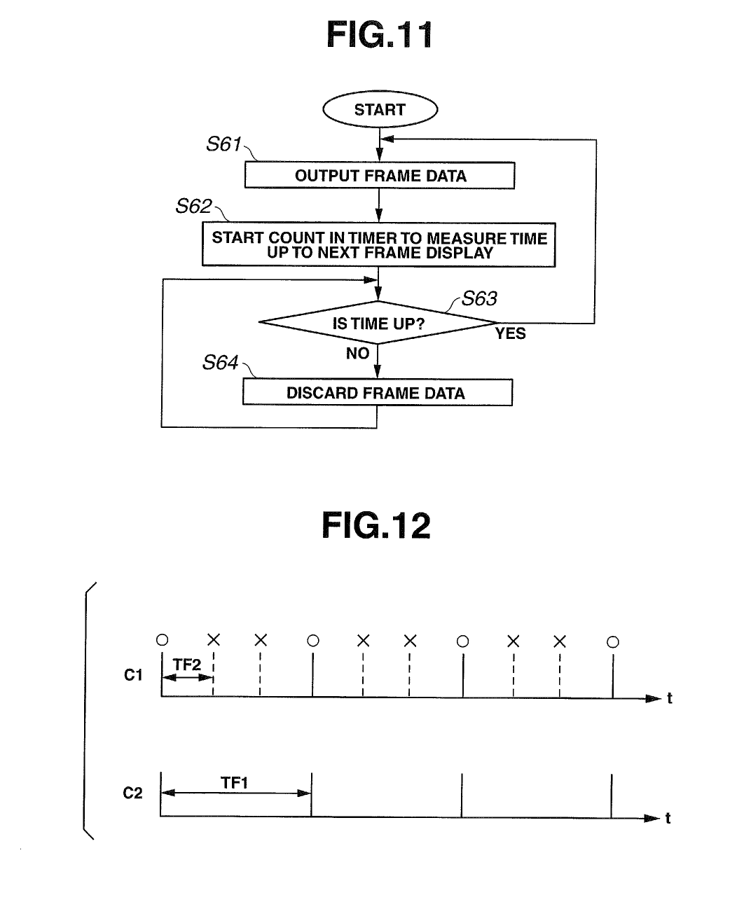

FIG. 11 is a flowchart showing details of frame data output processing in step S52 in FIG. 10. The video processing section 38 first outputs one-frame data generated in step S51 (step S61).

The video processing section 38 starts count in a timer to measure the time up to a next frame display (step S62). A value set in the timer is the value of the time corresponding to the display frame rate obtained by the above-described computation.

Determination is made as to whether or not time in the timer is up (step S63). If time is not up, that is, in the case of NO, frame data is discarded (step S64) and the process returns to step S63. That is, in the video processing section 38, frame data received before time in the timer is up is discarded.

When time in the timer is up, that is, in the case of YES in step S63, the process returns to step S61. The above-described processing is repeated to generate the image on the right-hand side of FIG. 19.

FIG. 12 is a diagram for explaining output and discarding of frame data by processing shown in FIGS. 10 and 11.

As shown in FIG. 12, in the electronic mode, a frame generation interval TF1 is determined when the display range is C2, and a frame generation interval TF2 is determined when the display range is C1 smaller than C2. For example, TF1 is a frame generation interval when the display range is 12 cm, while TF2 is a frame generation interval when the display range is 4 cm.

Under such a condition, the timer count value is set so as to measure the time corresponding to TF1, thereby discarding frame data outputted by timing indicated by symbol X, while outputting frame data by timing indicated by symbol ◯. That is, even when the display range is C1, frame data is outputted only by timing corresponding to C2.

In consequence, the video processing section 38 as control means or a control section controls output of frame data on ultrasound tomographic images from the graphic memory 37 storing image data on ultrasound tomographic images, so that the number of display frames of ultrasound tomographic images per stroke time in the manual scanning mode is constant even when the display range is changed.

According to the present embodiment, as described above, the same advantage as that of the first embodiment can be achieved. In particular, the present embodiment also has a merit in that only changing the software for the video processing section suffices.

### Third Embodiment

A third embodiment of the present invention will be described.

While in the above-described two embodiments the frame rate is set to a predetermined value, the frame rate is determined on the basis of a stroke time inputted or set by a user in the present embodiment. An ultrasound diagnostic apparatus in the third embodiment has the same hardware configuration as that of the ultrasound diagnostic apparatuses in the first and second embodiments. The same components as those of the ultrasound diagnostic apparatuses in the first and second embodiments are indicated by the same reference characters, and the description for the same components will not be repeated.

In the above-described two embodiments, the frame rate at the time of manual-draw scanning may be a value set in advance or a set value changeable by a user.

However, in a case where the position or area of a tumor portion is recognized by an operator as a result of making a first observation, and where only a region containing the particular portion is then observed, enabling input of a stroke time is convenient for the operator. For example, if the size of the tumor portion is found to be about ¼ of the whole as a result of the first observation with a stroke time of 12 seconds, it can be understood that the tumor portion can be observed in a magnified state when the stroke time is changed to ¼.

Then, in a case where a stroke time is set by an operator, the frame rate computed on the basis of the set stroke time is set as the above-described predetermined value.

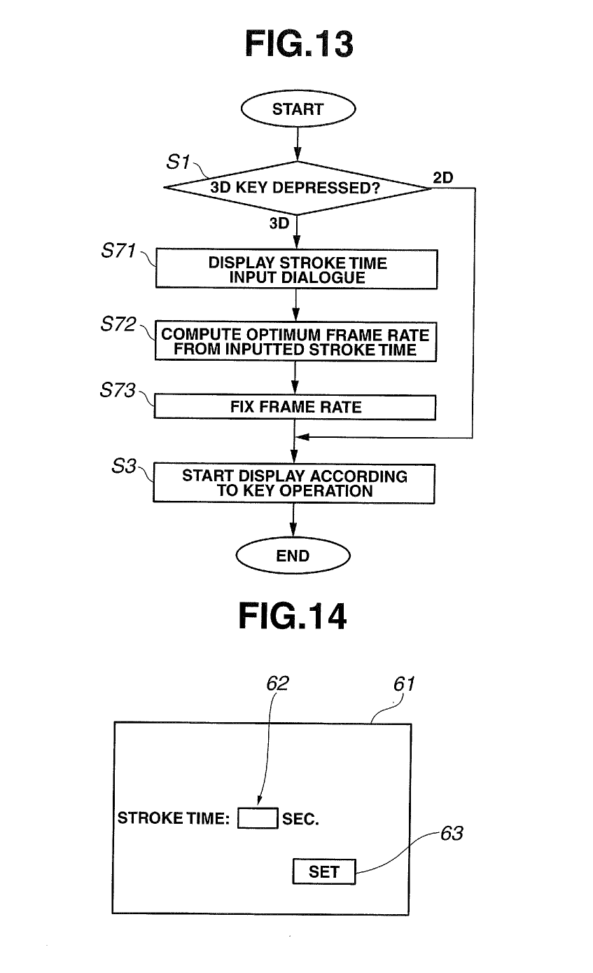

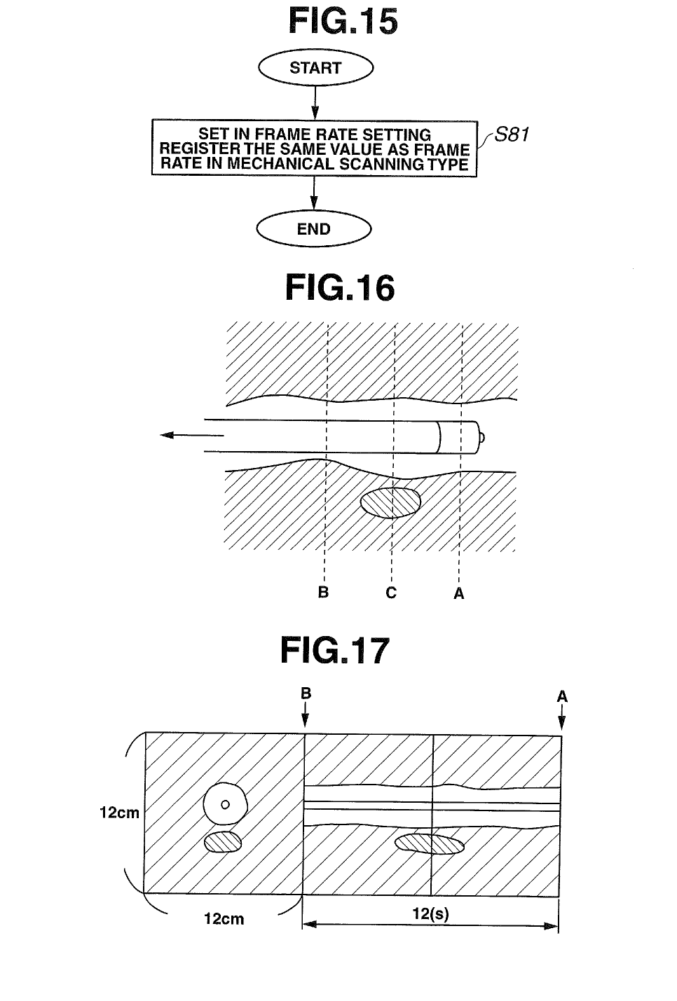

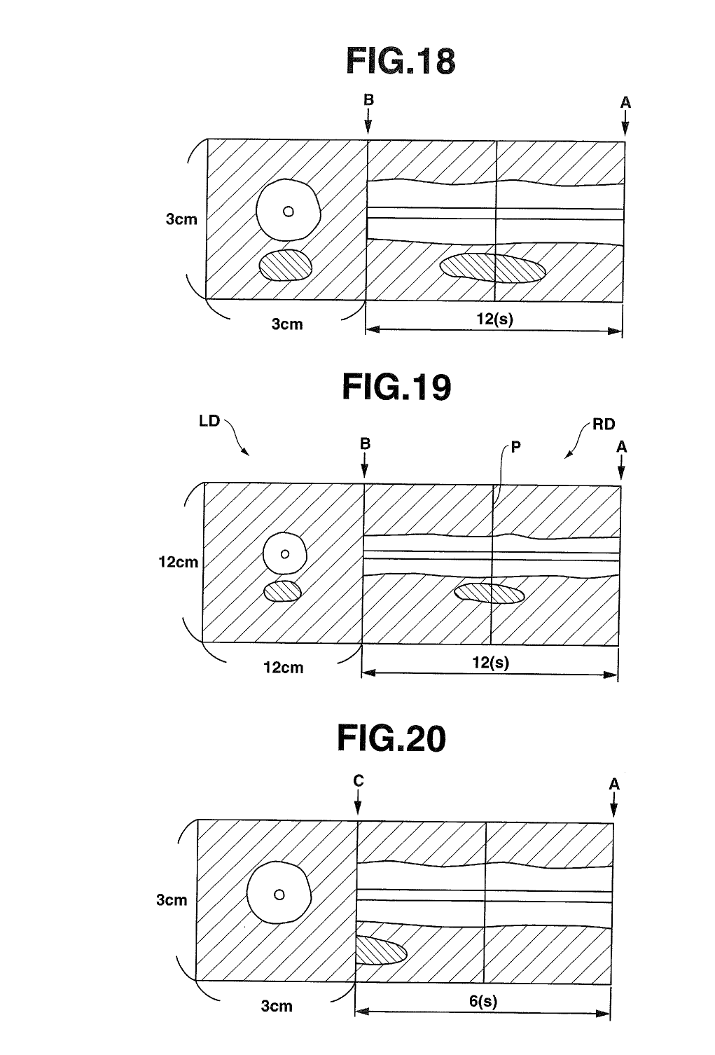

FIG. 13 is a flowchart showing an example of the flow of the entire processing in the ultrasound diagnostic apparatus 1 in the present embodiment. The frame rate is computed on the basis of an inputted stroke time. In FIG. 13, the same constituents as those in FIG. 3 are indicated by the same step numbers.

When the 3D key is depressed (step S1), the CPU 39a displays on the screen of the monitor 5 an input dialogue for input of a stroke time by a user (step S71).

FIG. 14 is a diagram showing an example of the input dialog for input of a stroke time. A user performs a predetermined operation to display an input dialogue view 61 shown in FIG. 14. The input dialogue view 61 may be a pop-up view or the like on the screen of the monitor 5. The user can set a desired stroke time by inputting the stroke time to an input field 62 and by clicking a setting button 63 on the screen.

The CPU 39a computes the frame rate from the inputted stroke time, thereby determining the stroke time (step S72).

Since the frame rate is frame rate=(number of frames/stroke time), the CPU 39a obtains the frame rate by computation. The frame rate obtained by computation may be used as a predetermined value without being changed or may be used after being changed to an optimum value in the vicinity of the obtained frame rate value.

The CPU 39a fixes the frame rate to the value obtained by computation (step S73) and the process advances to subsequent step S3. The frame rate is thus fixed in the case of the electronic scanning system.

According to the present embodiment, as described above, setting of a stoke time is enabled to enable manual-draw scanning at a speed according to a user's preference in the 3D display mode without requiring any complicated operation as well as to achieve the same advantages as those of the first and second embodiments.

While a stroke time is set in the above-described example, a stroke length proportional to a stroke time may be inputted instead of the stroke time.

### Fourth Embodiment

A fourth embodiment of the present invention will be described.

In the above-described first to third embodiments, processing when manual-draw scanning is performed by using an electronic-scanning-type ultrasound endoscope to generate three-dimensional data is independent of processing when manual-draw scanning is performed by using a mechanical-scanning-type ultrasound probe.

Also, the first and second embodiments have been described by way of example with respect to an ultrasound diagnostic apparatus in which two ultrasound apparatuses: a mechanical-scanning-type apparatus and an electronic-scanning-type apparatus are connected. However, the details of the processings described in the descriptions of the first and second embodiments can also be applied to an ultrasound observation apparatus to which a mechanical-scanning-type ultrasound probe cannot be connected, and in which only an electronic-scanning-type ultrasound endoscope can be used.

The present embodiment is arranged so that when an electronic-scanning-type apparatus is connected in an ultrasound diagnostic apparatus in which two ultrasound apparatuses: a mechanical-scanning-type apparatus and the electronic-scanning-type apparatus are connected, the above-described predetermined value is the same as the frame rate for the mechanical-scanning-type ultrasound probe. The ultrasound diagnostic apparatus according to the fourth embodiment has the same hardware configuration as that of the ultrasound diagnostic apparatuses in the first to third embodiments. The same components as those of the ultrasound diagnostic apparatuses in the first to third embodiments are indicated by the same reference characters, and the description for the same components will not be repeated.

FIG. 15 is a flowchart showing an example of the flow of processing for frame rate fixing control according to the present embodiment. As shown in FIG. 15, the CPU 39a sets in the frame rate setting register 39a the same value as the frame rate for the mechanical-scanning-type ultrasound probe (step S71).

In the first embodiment, this processing may be performed in place of the processing at the time of setting the predetermined value in the frame rate register shown in FIG. 4. In the second embodiment, processing shown in FIG. 14 may be performed in place of frame rate computation in step S52 in FIG. 10.

In consequence, control means or a control section such as the signal processing section performs control so that the numbers of display frames of ultrasound tomographic images respectively produced by the mechanical-scanning-type ultrasound probe and the electronic-scanning-type ultrasound endoscope or ultrasound probe are equal to each other.

According to the present invention, the same advantage as that of the first embodiment described above can be achieved. Moreover, when an operator performs manual-draw scanning for producing three-dimensional image data by using an electronic-scanning-type ultrasound endoscope, the operator performs scanning at the same drawing speed as that when manual-draw scanning is performed by using a mechanical-scanning-type ultrasound probe. The present embodiment therefore also has a merit in that a user is free from a feeling of unnaturalness in operatively even when the ultrasound apparatus is changed.

According to each of the embodiments, as described above, three-dimensional image data is produced in an electronic-scanning-type ultrasound observation apparatus. As a result, an ultrasound observation apparatus requiring no complicated manual scanning while eliminating the need for an expensive apparatus and avoiding an increase in probe diameter can be realized.

The present invention is not limited to the above-described embodiments. Various changes and modifications can be made in the embodiments without departing from the gist of the present invention.

1. An ultrasound observation apparatus which has an ultrasound probe or an ultrasound endoscope manually moved relative to a subject, and which displays a plurality of ultrasound tomographic images in time sequence with the movement,
the apparatus comprising a control section which, when a first display range is selected, performs control so that images are displayed in a first number of displayed frames per the stroke time, which, when a second display range is selected, performs control so that the number of displayed frames per the stroke time is smaller than the first number of displayed frames, and which, when a manual scanning mode is selected, performs control so that a predetermined number of frames per the stroke time are displayed regardless of whether the display range is the first display range or the second display range.

2. The ultrasound observation apparatus according to claim 1, wherein the control section performs control so that the number of displayed frames per the stroke time of the ultrasound tomographic images is made constant by generating a frame sync signal on the basis of a set predetermined value.

3. The ultrasound observation apparatus according to claim 2, wherein the predetermined value is a number of frames or a period corresponding to the number of frames.

4. The ultrasound observation apparatus according to claim 1, wherein the control section performs control so that the number of displayed frames per the stroke time of the ultrasound tomographic images is made constant by controlling output of frame data on ultrasound tomographic images generated from a graphic memory storing image data on the ultrasound tomographic images.

5. The ultrasound observation apparatus according to claim 1, wherein a setting of the stroke time can be made.

6. The ultrasound observation apparatus according to claim 5, wherein a stroke length proportional to the stroke time is set in place of the stroke time.

7. The ultrasound observation apparatus according to claim 5, wherein a setting of the stroke time can be made through a view generated for setting of the stroke time.

8. The ultrasound observation apparatus according to claim 1, wherein the ultrasound probe or the ultrasound endoscope is an electronic-scanning-type ultrasound probe or ultrasound endoscope.

9. The ultrasound observation apparatus according to claim 1,
wherein a mechanical-scanning-type ultrasound probe or ultrasound endoscope and an electronic-scanning-type ultrasound probe or ultrasound endoscope can be connected to the ultrasound observation apparatus, and
wherein the control section performs control so that the numbers of displayed frames of the ultrasound tomographic images respectively produced by the mechanical-scanning-type ultrasound probe or ultrasound endoscope and the electronic-scanning-type ultrasound probe or ultrasound endoscope are equal to each other.

10. The ultrasound observation apparatus according to claim 9, further comprising:
a first connection sensing section which senses a connection of the mechanical-scanning-type ultrasound probe or ultrasound endoscope; and
a second connection sensing section which senses a connection of the electronic-scanning-type ultrasound probe or ultrasound endoscope.

11. The ultrasound observation apparatus according to claim 9, wherein the control section performs control so that the number of displayed frames of ultrasound tomographic images produced by the electronic-scanning-type ultrasound probe or ultrasound endoscope is the same as the number of displayed frames of ultrasound tomographic images produced by the mechanical-scanning type ultrasound probe or ultrasound endoscope.

12. The ultrasound observation apparatus according to claim 2, wherein the control section performs control so that the number of displayed frames per the stroke time of the ultrasound tomographic images is made constant by controlling output of frame data on ultrasound tomographic images generated from a graphic memory storing image data on the ultrasound tomographic images.
